(19) United States
(12) Patent Application Publication
(10) Pub. No.: US 2026/0115031 A1
(43) Pub. Date: Apr. 30, 2026

(54) ADJUSTABLE TOE SEPARATOR DEVICE

(71) Applicant: Kusha Karvandi, Scottsdale, AZ (US)

(72) Inventor: Kusha Karvandi, Scottsdale, AZ (US)

(21) Appl. No.: 19/019,862

(22) Filed: Jan. 14, 2025

Related U.S. Application Data
(63) Continuation of application No. 29/969,892, filed on Oct. 24, 2024, now Pat. No. D1064300.

(51) Int. Cl. A61F 5/01; A63B 23/10

(52) U.S. Cl. CPC A61F 5/019; A63B 23/10

(57) ABSTRACT

A toe separator device provides a plurality of spacer pillars for separating the second, middle, fourth and little toes. The toe separator device isolates the big toe in a spacer shell that is slidably received therein. The spacer shell provides a wedge volume disposed between the big toe and the second toe, wherein the wedge volume has an adjustment slot for slidably received an separator pill to selectively determine the width of the wedge volume between the big toe and the second toe.

### BACKGROUND OF THE SUBJECT DISCLOSURE

The present invention relates to toe separators and, more particularly, to an adjustable toe separator device adapted for being worn while the user is on their feet and active.

Healthy feet are widest at the toes. However, conventional shoes, which most people have been wearing since childhood, feature a narrow toe box (the part of the shoes where your toes sit). A lifetime of squishing feet into narrow shoes causes the actual shape of the feet to change, and so causing dysfunction iFn foot mechanics, mobility, posture, and physical fitness. Specifically, when toes point inwards, the connective tissue of our feet shortens, circulation is restricted, and the formation of bunions is not uncommon.

Reclaiming foot health involves, among other things, wearing toe separators to assist the foot in returning to its natural shape, again, widest at the toes since the long-term benefits of proper toe spacing include increased circulation, improved balance, and stronger feet.

However, just as one shoe does not fit every foot, no single toe separator fits every set of toes. Current toe separator devices that are not adjustable do not work well because the user often finds the device too hard, too soft, too small, too tight, or too loose.

Moreover, most toe spacer devices are restrictive and cumbersome to put on, since many do not enable the user to adjust the thickness of the spacer between toes. And for those toe separator devices that use adjustable implements, such adjustment implements are insufficiently secured and easily come out when the user walks or is otherwise physically active and on their feet. In short, present toe spacer devices are not adapted for wearing during walking or exercise.

Currently toe spacer devices have poor user compliance due to discomfort and ineffectiveness. This ineffectiveness stems directly from these devices not being able to provide customizable separation for the big toe, which will yield less favorable results since the toe spacer devices are only functional when the user is seated or stationary.

As can be seen, there is a need for an adjustable toe separator device adapted for being worn while the user is on their feet and active.

### SUMMARY OF THE SUBJECT DISCLOSURE

The toe separator device embodied in the subject disclosure is easy to put on and take off and is securely adjustable by way of a separator pill. Furthermore, the toe separator device is configured so that the separator pill stays in place even while the device is worn while the user is walking or exercising, which is ideal for the foot and toes to adapt and see improvement faster.

The toe separator device utilizes a removable and interchangeable toe separator pill system, wherein the toe separator device isolates the big toe in a separator shell design. The separator shell has a wedge volume disposed between the big toe and the second toe, wherein the wedge volume provides an adjustment slot that a separator pill may be inserted, thereby selectively widening the width of each toe wedge. The toe separator pills available come in different densities to provide an even further level of customization for the user.

In one aspect of the present subject disclosure, a toe separator device provides the following: a base having a first cross-sectional width and a second cross-sectional width; a plurality of spacer pillars extending from the first cross-sectional width; a closed loop spacer shell extending from the second cross-sectional width, wherein the closed loop spacer shell defines an enclosed hole configured to receive a big toe of a user; a wedge volume along a medial portion of the spacer shell; and an adjustment pill slot in the wedge volume.

In another aspect of the present subject disclosure, the toe separator device further includes wherein each two adjacent spacer pillars of the plurality of spacer pillars defines an inverted trapezoidal prism void, and wherein a medial face of the wedge volume is angled relative to the base to define another trapezoidal prism void relative to the spacer pillar of the plurality of spacer pillars associated with the second toe, wherein the adjustment pill slot is substantially parallel with the angled medial face; further providing an adjustment pill dimensioned and shaped to be removably inserted into the adjustment pill slot, wherein the adjustment pill is fish-shaped including a caudal fin portion; further providing a taper between the first and second cross-sectional widths of the base, wherein the difference between the first and second cross-sectional widths is at least a twenty-five percent increase, and wherein each of the plurality of spacer pillars and the closed loop spacer shell have a respective upper surface that are coplanar.

These and other features, aspects and advantages of the present subject disclosure will become better understood with reference to the following drawings, description and claims.

### BRIEF DESCRIPTION OF THE DRAWINGS

FIG. 1 is a front perspective view of an exemplary embodiment of the subject disclosure.

FIG. 2 is a detailed perspective view of an exemplary embodiment of the subject disclosure.

FIG. 3 is a front elevation view of an exemplary embodiment of the subject disclosure.

FIG. 4 is an exploded perspective view of an exemplary embodiment of the subject disclosure.

FIG. 5 is a perspective view of an exemplary embodiment of the subject disclosure.

FIG. 6 is a perspective view of an exemplary embodiment of the subject disclosure, wherein a wide embodiment 20A of the closed loop spacer shell and thus wide embodiment of separator pills 20B.

### DETAILED DESCRIPTION OF THE SUBJECT DISCLOSURE

The following detailed description is of the best currently contemplated modes of carrying out exemplary embodiments of the subject disclosure. The description is not to be taken in a limiting sense but is made merely for the purpose of illustrating the general principles of the subject disclosure, since the scope of the subject disclosure is best defined by the appended claims.

Broadly, an embodiment of the present subject disclosure provides a toe separator device made from a flexible or semi-flexible material such as rubber or silicone. The toe separator device provides a plurality of spacer pillars for separating the second, middle, fourth and little toes. The toe separator device isolates the big toe in a spacer shell that is slidably received therein. The spacer shell provides a wedge volume disposed between the big toe and the second toe, wherein the wedge volume has an adjustment slot for slidably received a separator pill to selectively determine the width of the wedge volume between the big toe and the second toe.

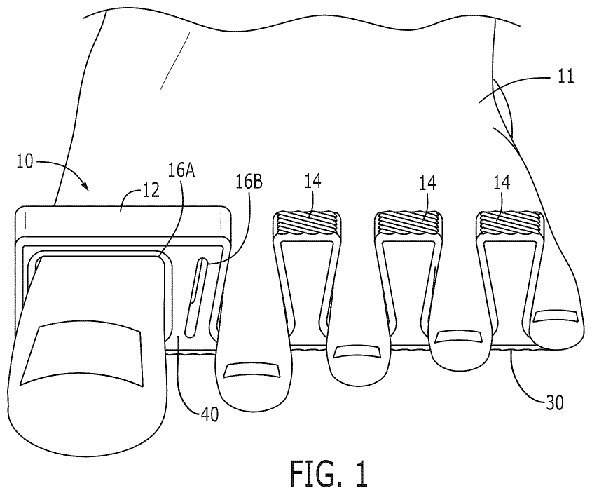

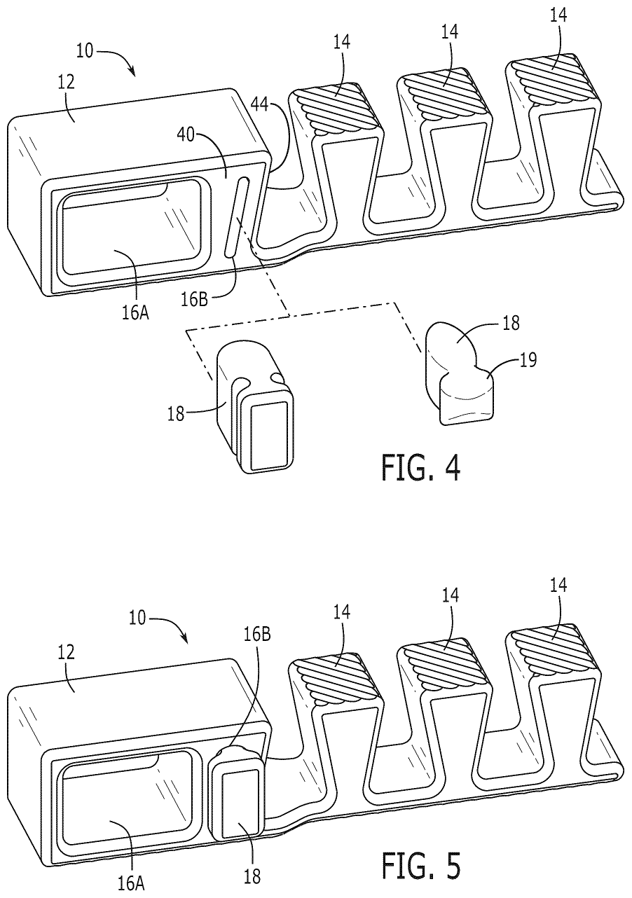

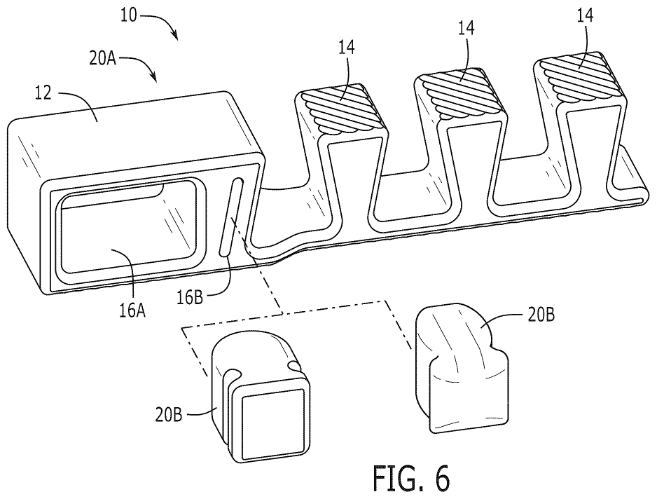

Referring to FIGS. 1 through 6, an embodiment of the subject disclosure provides a toe separator device 10. The toe separator device 10 has a base 30 that extends under the toes of a user 11 when the toe separator device 10 is worn, as illustrated in FIG. 1. Projecting “upward” (when the toe separator device 10 is worn and the user is standing upright) from an upper surface of the base 30 is a big toe spacer shell 12 for receiving the big toe of the user 11 and three spacer pillars 14 for receiving the remaining “non-big” second, middle, fourth and little toes.

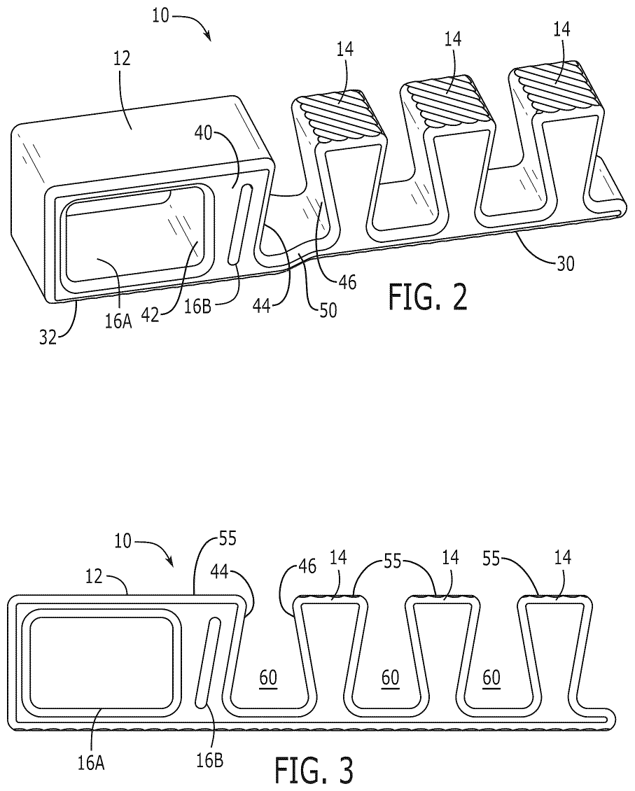

Critically, the cross-sectional width of the base 30 increases as it transitions from supporting the three spacer pillars 14 to supporting the closed-loop spacer shell 12, as denoted by reference numeral 50 in FIG. 2, which indicates this transition or taper from the base portion associated with the three spacer pillars 14 and the (second) base portion 32 associated with the closed-loop spacer shell 12. The increase in cross-sectional width of the base 30 is at least a twenty-five percent increase. The increase in cross-sectional width, in conjunction with the closed-loop spacer shell 12 facilitates the stability a user 11 feels when standing or otherwise being active ‘on the feet’ while using the device embodied in the subject disclosure. The first cross-sectional width should be approximately 0.25 inches, while the second cross-sectional width should be approximately 0.50 inches.

Also important, the upper surfaces 55 (see FIG. 3) of the closed-loop spacer shell 12 and the three spacer pillars 14 have a ‘top-off’ elevation that is substantially co-planar and of a substantial area (see below regarding the top trapezoidal bases of the three spacer pillars 14) to help facilitate lateral support for the toe separator device 10 while worm by the user who is walking or otherwise actively moving on their feet.

The three spacer pillars 14 are dimensioned and shaped to be received in the gaps of the four non-big toes of the user 11, as illustrated in FIG. 1. Each spacer pillar 14 may have a substantially trapezoidal prism shape that is defined by an isosceles trapezoidal cross-section (when cut along a coronal plane of the FIG. 3), wherein the top trapezoidal base has a surface area that is at least twice that of the surface area of the bottom trapezoidal base.

As a corollary, the voids 60 of ‘toe slots” between adjacent spacer pillars 14 are “inverted trapezoidal prisms” (an inversion of the trapezoidal prism of a spacer pillar 14), wherein the top trapezoidal base of such voids 60 is less than half that of its bottom trapezoidal base. This wedge-like shape of the spacer pillar 14 facilitates providing space between adjacent non-big toes.

The closed-loop spacer shell 12 provides a big toe hole 16A through which the big toe of the user 11 is received. The closed-loop spacer shell 12 also provides—between the big toe and the second toe (i.e., the index toe or pointer toe) a wedge volume 40. The wedge volume 40 is defined, on one end, by the medial wall 42 of the big toe hole 16A and on an opposing end by a medial face 44 thereof. The medial face 44 complements the adjacent face 46 of the adjacent spacer pillar 14 to define an inverted trapezoidal prism void for the second toe, like the void defined between two adjacent spacer pillars 14.

The wedge volume 40 also defines a pill slot 16B that is substantially parallel with the medial face 44. The pill slot 16B is dimensioned and shaped to removably receive a toe separator pill 18. The subject disclosure contemplates various separator pills of different shapes, sizes and densities so that a user 11 can selectively adjust the resulting width and form factor of the wedge volume 40 based on the comfort level and desired foot functioning improvement. In some embodiments, the distal end (the posterior end) of the toe separator pill 18 may protrude from the pill slot 16B. The first separator pill 18 may be fish-shaped, having a body portion and a caudal fin portion 19. All or a portion of the caudal fin portion may protrude from the pill slot 16B during use.

Once the toe spacer pill 18 is inserted into the adjustment pill slot 16B of the closed-loop spacer shell 12, the closed-loop spacer shell 12 may be put onto the user's 11 foot beginning with their big toe inside and through the big toe hole 16A, and then the other toes set between the toe spacer pillars 14.

The user could then go about their normal daily activities with the device on their feet while standing, walking or even exercising while the device helps correct their feet to have the toes splayed more naturally. The triangular/trapezoidal prism shape of the spacer pillars 14 and the closed loop on the big toe as well as the tapering 50 increase of the cross-sectional width of the base is critical to enabling beneficial properties of the subject disclosure while the user 11 is walking or otherwise on their feet.

As used in this application, the term “about” or “approximately” refers to a range of values within plus or minus 10% of the specified number. And the term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down,” and the like, are words of convenience and are not to be construed as limiting terms unless specifically stated to the contrary.

It should be understood, of course, that the foregoing relates to exemplary embodiments of the subject disclosure and that modifications may be made without departing from the spirit and scope of the subject disclosure as set forth in the following claims.

What is claimed is:

1. A toe separator device comprising:
a base having a first cross-sectional width and a second cross-sectional width;
a plurality of spacer pillars extending from the first cross-sectional width;
a closed loop spacer shell extending from the second cross-sectional width, wherein the closed loop spacer shell defines an enclosed hole configured to receive a big toe of a user;
a wedge volume along a medial portion of the spacer shell; and
an adjustment pill slot in the wedge volume.

2. The toe separator device of claim 1, wherein each two adjacent spacer pillars of the plurality of spacer pillars defines an inverted trapezoidal prism void, and wherein a medial face of the wedge volume is angled relative to the base to define another trapezoidal prism void relative to the spacer pillar of the plurality of spacer pillars associated with the second toe.

3. The toe separator device of claim 2, wherein the adjustment pill slot is substantially parallel with the angled medial face.

4. The toe separator device of claim 3, further comprising an adjustment pill dimensioned and shaped to be removably inserted into the adjustment pill slot.

5. The toe separator device of claim 4, wherein the adjustment pill is fish-shaped including a caudal fin portion.

6. The toe separator device of claim 3, further comprising a taper between the first and second cross-sectional widths of the base.

7. The toe separator device of claim 6, wherein the difference between the first and second cross-sectional widths is at least a twenty-five percent increase.

8. The toe separator device of claim 7, wherein each of the plurality of spacer pillars and the closed loop spacer shell have a respective upper surface that are coplanar.
